(19) United States
(12) Patent Application Publication
(10) Pub. No.: US 2022/0357309 A1
(43) Pub. Date: Nov. 10, 2022

(54) METHODS FOR DISTINGUISHING THE STAGES OF BACTERIAL VAGINOSIS

(71) Applicant: University Of Maryland, Baltimore, Baltimore, MD (US)

(72) Inventors: REBECCA BROTMAN, Baltimore, MD (US); JACQUES RAVEL, Laurel, MD (US); D. ELIZABETH O'HANLON, Baltimore, MD (US)

(21) Appl. No.: 17/774,215

(22) PCT Filed: Nov. 4, 2020
(86) PCT No.: PCT/US2020/058893

§ 371 Date: May 4, 2022

Related U.S. Application Data
(60) Provisional application No. 62/930,147, filed on Nov. 4, 2019.

(51) Int. Cl. G01N 33/483; G01N 1/30; G01N 21/88

(52) U.S. Cl. CPC G01N 33/4833; G01N 1/30; G01N 21/8851; G01N 2001/302; G01N 2800/36

(57) ABSTRACT

This application provides a method for staging bacterial vaginosis (BV), i.e., determining whether a subject does not have BV, has Stage I BV, or has Stage II BV. The method relies on testing for the number and maturity of shed epithelial cells from a sample of vaginal secretion from the subject. Specific diagnosis of Stage I or Stage II BV allows the clinician to provide a targeted and appropriate treatment or treatment course for the subject.

### CROSS-REFERENCE TO RELATED APPLICATIONS

This application is a National Stage entry of and claims priority to PCT/US2020/058893, filed 4 Nov. 2020, which claims the benefit of United States Provisional Application Ser. No. 62/930,147, filed 4 Nov. 2019. The entire contents of these applications are hereby incorporated by reference as if fully set forth herein.

### GOVERNMENT FUNDING SUPPORT

This invention was made with government support under grant no. AI119012 awarded by the National Institutes of Health. The government has certain rights in the invention.

### BACKGROUND

### 1. Field of the Invention

The present invention relates to the field of medicine and specifically to methods for distinguishing the stages of bacterial vaginosis (BV) in subjects based on the number and maturity of shed epithelial cells. The methods described herein can be used for development of a point-of-care bacterial vaginosis diagnostic and allows a targeted treatment regimen for treating BV as appropriate to the stage of BV.

### 2. Background of the Invention

Bacterial vaginosis is a very common condition in which the cervicovaginal microbiota is composed predominantly of mixed anaerobes. BV significantly increases a woman's risk of acquiring a sexually transmitted infection and may increase the risk of adverse pregnancy outcomes. In the past, BV has been categorized as symptomatic or asymptomatic, based on subjective patient perception of symptoms (vaginal discharge; pain, itching, or burning; strong odor; burning upon urination) and the presence of signs known as Amsel's criteria. Amsel's criteria are (1) increase homogenous thin vaginal discharge; (2) pH of the secretion greater than 4.5; (3) amine odor when KOH 10% solution is added to a drop of vaginal secretions; and (4) the presence of clue cells in wet preparations. The presence of three of these criteria indicates a diagnosis of BV, but does not enable the clinician to determine the stage of the condition or distinguish between symptomatic BV (sBV) and asymptomatic (aBV).

Clinically, BV is not usually treated if asymptomatic (i.e., no uncomfortable discharge, odor, itching, and the like) and, indeed, does not seem to respond well to current treatment. BV can also be diagnosed using microscopy and standardized scoring (known as Nugent scoring) of the numbers and morphologies of bacteria in a collected vaginal sample. This method is widely used in research settings but also does not distinguish between stages of the condition. There is no molecular diagnostic specific for symptomatic and asymptomatic that can determine the stage of BV.

### SUMMARY OF THE INVENTION

Therefore, there is a need in the art for an objective method of diagnosing and distinguishing the stages of BV for clinical use. The present study involved using a collection of cervicovaginal smears for which microbiota composition and clinical data were available in order to show that the number and maturity of shed epithelial cells observed in the different stages of BV differ so that this can be used in diagnostic modalities. A BV diagnostic test or system that was able to differentiate these stages BV would be superior to any in current use.

In particular, the present invention relates, in some embodiments to a method of diagnosing the stage of bacterial vaginosis in a subject, comprising: (a) obtaining a sample of vaginal secretion from the subject; (b) determining the total number of shed epithelial cells in the sample; and (c) determining the maturity of shed epithelial cells in the sample to obtain epithelial cell counts for superficial cells and for parabasal cells in the sample to diagnose Stage I or Stage II of bacterial vaginosis, wherein Stage I bacterial vaginosis is diagnosed when total cell shedding log10 cell count is from about 2.4 to about 3.0, superficial cells log10 cell count is from about 1.5 to about 2.7 and parabasal cells log10 cell count is from about 1.1 to about 2.5; and wherein Stage II bacterial vaginosis is diagnosed when total cell shedding log10 cell count is from about 1.5 to about 2.5, superficial cells log10 cell count is about 1.0 to about 1.7 and parabasal cells log10 cell count is about 1.2 to about 2.3.

The invention also relates, in certain embodiments, to a diagnostic kit for diagnosing the stage of bacterial vaginosis comprising means for performing the method described herein.

In other embodiments, the invention relates to a method of treating bacterial vaginosis in a subject in need, comprising: (a) performing the method of claim 1; and (b) treating the subject for bacterial vaginosis if the subject is diagnosed with Stage I BV and treating the subject for bacterial vaginosis if the subjects is diagnosed with Stage II BV. In these methods, the subject can be symptomatic or asymptomatic.

### BRIEF SUMMARY OF THE DRAWINGS

FIG. 1 is a summary graph showing the proportion of CST cells in each of the samples, characterized according to the system in current use: aBV, sBV, and noBV samples.

FIG. 2 is a graph illustrating a course of BV disease where stage I is followed stage II fairly rapidly.

FIG. 3A through FIG. 3D are sets of bright-field photomicrographs (100× total magnification). FIG. 3A is a bright-field micrograph showing a sample with no BV as classified by Amsel criteria. FIG. 3B is a bright-field micrograph showing a BV sample as determined by Amsel criteria. FIG. 3C is a bright-field micrograph showing a sample with no BV as classified by Amsel criteria with a cell count. FIG. 3D is a bright-field micrograph showing a BV sample classified by Amsel criteria in an asymptomatic subject with a cell count. Scale bars represent 100 μm.

FIG. 4A is a box-percentile plot of log10 mean epithelial cell counts in different Amsel-classified diagnostic groups.

FIG. 4B is a box-percentile plot of log10 mean cell-aggregate sizes in different Amsel-classified diagnostic groups. The median, 25th, and 75th percentiles are marked with line segments across each box. Numbers at the top axis indicate the number of samples in each group. Ticks within percentile-boxes show individual sample values.

FIG. 5A is a bright-field micrograph (100× total magnification) showing sample with single cells and small cell-aggregates.

FIG. 5B is a photograph without magnification showing samples with cell-aggregates large enough to see with the naked eye.

FIG. 6 is a box-percentile plot of superficial cell index in no BV and BV (asymptomatic) samples by Amsel criteria, with a mean epithelial cell count of less than 50.

FIG. 7A is a box-percentile plot of log10 mean epithelial cell counts in different diagnostic groups for only CST IV samples. The dashed line indicates 50 mean epithelial cells.

FIG. 7B is a box-percentile plot of log₁₀ mean cell-aggregate sizes in different diagnostic groups for only CST IV samples. The median, 25th and 75th percentiles are marked with line segments across each box. Numbers at the top axis indicate the number of samples in each group. Ticks within percentile-boxes show individual sample values.

FIG. 8 is a box-percentile plot of superficial cell index in no Amsel-BV and asymptomatic Amsel-BV samples for only CST IV samples with a mean epithelial cell count of less than 50.

FIG. 9 is a box-percentile plot of log10 mean epithelial cell counts in noBV samples assigned to CST IV or Lactobacillus CSTs (I, II, III or V). The median, 25th, and 75th percentiles are marked with line segments across each box. Numbers at the top axis indicate the number of samples in each group. Ticks within percentile-boxes show individual sample values.

FIG. 10 is a Table of cell counts, cell aggregate sizes, SCI and metadata for the samples (n=192) used in this study.

### DETAILED DESCRIPTION

### 1. OVERVIEW

The cervicovaginal microbiota is known to contribute to a woman's protection against and susceptibility to reproductive tract infections (RTls). Women whose cervicovaginal microbiota is composed predominantly of Lactobacillus spp. are at decreased risk of many sexually transmitted infections, including HIV (Taha et al., 1998; Gosmann et al.,2017), HPV (Mitra et al., 2016), HSV-2 (Cherpes et al.,2003), trichomoniasis (Brotman et al.,2012), gonorrhea and chlamydia (Wiesenfeld et al., 2003) compared to women with microbiota comprising strict and facultative anaerobic bacteria. The latter is what broadly characterizes bacterial vaginosis (BV), a condition that is defined differently in clinical and research settings (McKinnon et al.,2019). Clinically, Amsel-BV (Amsel et al., 1983) is characterized by the presence of certain clinical signs with patients reporting symptoms (symptomatic BV (sBV)) or no symptoms (asymptomatic BV (aBV)) on direct questioning (Fleury,1983; Eschenbach et al.,1988).

Vaginal community state types (CSTs) have been used to classify the states of vaginal microbial communities. See Table 1, below. This is a qualitative classification of bacterial types useful for interpreting BV etiology. In research studies, “Nugent-BV” is characterized, through microscopic examination of a vaginal smear, by a predominance of Gram-negative Gardnerella and Bacteroides spp. morphotypes rather than Gram-positive Lactobacillus spp. morphotypes (Nugent et al.,1991). Vaginal microbiota assessment using culture-independent approaches based on the quantification and characterization of bacterial 16S rRNA gene sequences defines “molecular-BV” when a microbiota both lacks high relative abundance of Lactobacillus spp. and is composed predominantly of a wide array of strict and facultative anaerobic bacteria (CST IV) (Gajer et al., 2012). Neither Nugent-BV nor molecular-BV testing can distinguish between stages of BV or determine whether the subject has a high or lower risk of infection. Although the concordance among the methods is not complete, it is generally true that Amsel-diagnosed BV, Nugent-diagnosed BV, and molecular-diagnosed BV are progressively more inclusive: in a given population, more women are positive by Nugent testing than for Amsel-based testing, and more are positive by molecular diagnosis than by Nugent diagnosis (McKinnon et al., 2019).

TABLE 1
Vaginal Community State Types/Classifications.

| CST | Dominant Species |
|---|---|
| I | L. crispatus |
| II | L. gasseri |
| III | L. mers |
| IV | N/A (diverse) |
| V | L. jensenii |

The cervicovaginal epithelium is the site at which multiple defenses against sexually transmitted infections (STls) and non-sexually transmitted reproductive tract infections (RTls) are deployed (Hickey et al.,2011). Lactobacillus spp. in the vaginal tract contribute to: (1) physical defenses, including mucus-trapping of pathogens (Nunn et al., 2015), suppression of inflammatory de-keratinization of epithelial cells (Zarate et al., 2009) and control of cell proliferation often necessary for infection (Edwards et al., 2019); (2) immunological defenses, including suppression of pro-inflammatory signaling through toll-like receptors (Mirmonsef et al., 2011) and suppression of pro-inflammatory cytokine expression (Jespers et al., 2017); and (3) biochemical defenses, including inducing expression of protective peptides (Yarbrough et al., 2015) and production of lactic acid (Tachedjian et al., 2017).

The homeostatic balance between the proliferation/maturation and shedding/loss of cells on the vaginal epithelium is likely critical in maintaining these defenses. Balanced proliferation/maturation and shedding/loss means the luminal surface consists of dead detaching cells and the adherence of pathogens to these sloughing cells has been hypothesized as a mechanism to provide some protection to the underlying living cells, which are vulnerable to productive infection (Anderson et al., 2014). Accelerated shedding/loss of cells from the vaginal epithelium, therefore, can be expected to decrease this protective function and thus increase susceptibility to STls and RTls. The composition of the cervicovaginal microbiota can affect the proliferation/maturation and shedding/loss balance: approximately twice as many shed epithelial cells were found in a study of vaginal smears from women with Nugent-diagnosed BV, versus women with predominantly Lactobacillus spp. microbiota (Amegashie et al., 2017).

Additionally, the composition of the cervicovaginal microbiota has been shown to control the expression of the host microRNA miR-193b which reduces cell proliferation (Edwards et al., 2019). Cell proliferation was lower in women with a BV-associated microbiota (Edwards et al., 2019), as were host proteins associated with epithelial maturation (Zevin et al., 2016). Further supporting this finding are observations of fewer mature and more immature epithelial cells in cervicovaginal samples from women lacking Lactobacilli (Fowler, 2012). At the same time, increased cell-shedding can eliminate non-Lactobacillus spp. bacteria attaching to the epithelium, such as Gardnerella vaginalis or Atopobium vaginae, which can form resilient biofilms (Hardy et al.,2016).

Typically, Amsel-diagnosed BV is treated only when symptomatic (Workowski and Bolan, 2015); however, inflammatory changes on the cervicovaginal epithelium are found in women with Nugent-BV (Thurman et al., 2015; Jespers et al., 2017) and molecular-BV (Anahtar et al., 2015), and so it is not surprising that the increased susceptibility to infections associated with BV also accrues to women with Nugent-BV (Cherpes et al., 2003; Wiesenfeld et al., 2003) and molecular-BV (Brotman et al., 2012; Brotman et al.,2014; Gosmann et al.,2017). Thus, Amsel-diagnosed BV in an asymptomatic subject still is problematic and should be treated in order to avoid a higher risk of STI and RTI in the subject. Now, it is possible to determine the stage of BV (and thus the risk of infection) and thereby to determine whether and how to treat the subject's specific condition.

### 1. Definitions

Unless defined otherwise, all technical and scientific terms used herein have the same meaning as commonly understood by one of ordinary skill in the art. Although various methods and materials similar or equivalent to those described herein can be used in the practice or testing of the present invention, suitable methods and materials are described below. However, the skilled artisan understands that the methods and materials used and described are examples and may not be the only ones suitable for use in the invention. Moreover, as measurements are subject to inherent variability, any temperature, weight, volume, time interval, pH, salinity, molarity or molality, range, concentration and any other measurements, quantities or numerical expressions given herein are intended to be approximate and not exact or critical figures unless expressly stated to the contrary.

As used herein, the term “about” means plus or minus 20 percent of the recited value, so that, for example, “about 0.125” means 0.125±0.025, and “about 1.0” means 1.0±0.2.

As used herein, the term “BV” refers to bacterial vaginosis, a condition characterized by an abnormal vaginal discharge due to an overgrowth of normal bacteria in the vagina.

As used herein, the term “symptomatic BV” refers to BV accompanied by signs and symptoms, including one or more of grayish or white vaginal discharge, watery discharge, odor, burning sensation while urinating, itching in the vulva area, and the like.

As used herein, the term “asymptomatic BV” refers to BV which is not accompanied by such symptoms listed above for symptomatic BV.

As used herein, the term “Stage I BV” is an early stage of BV which meets three of the four Amsel criteria and is accompanied by a subjective report of symptoms.

As used herein, the term “Stage II BV” is a later stage of BV defined by meeting three of the four Amsel criteria and is not accompanied by a subjective report of symptoms

As used herein, the term “CST” refers to community state types, which are the predominant species of bacteria present in the vaginas of women with BV.

As used herein, the term “SCI” refers to a superficial cell index, the number of superficial cells/number of all epithelial cells, which is a a measure of cervicovaginal epithelial cell maturity.

As used herein, the term “probiotic” refers to a live bacterial substance that are believed to provide health benefits by improving or restoring natural flora.

As used herein, the term “prebiotic” refers to a compound in food that induce the growth or activity of beneficial microorganisms.

As used herein, the term “symbiotic” refers to bacteria living in close physical association, typically to the advantage of both.

As used herein, the term “antibiotic” refers to a drug or compound that is used to treat bacterial infections, i.e., has the ability to kill or inhibit bacteria.

### 2. SUMMARY OF RESULTS

Bacterial vaginosis is associated with increased cell-shedding from the cervicovaginal epithelium. Cell-shedding in excess of cell-proliferation can decrease epithelial barrier function and increase susceptibility to infection. This study has evaluated the number of shed cells in mid-vaginal smears from women with a diagnosis of “symptomatic BV” (sBV, n=17), “asymptomatic BV” (aBV, n=71), or no BV (n=104) by Amsel diagnosis criteria. Smears taken from women diagnosed with sBV contained significantly more shed cells (median 158/100× field) than no BV smears (median 91/100× field), p=7.2e-9. However, we observed that smears from asymptomatic women diagnosed with BV by Amsel criteria contained significantly fewer shed cells (median 35/100× field) than no BV smears, p=22.0e-16. The sizes of cell-aggregates (cells shed in sometimes multilayered sections with intact cell-cell attachments) followed the same pattern. Cell-aggregates in sBV smears were significantly larger (median approximately 220,000 μm²) than those in no BV smears (median approximately 50,000 μm²), p=1.8e-6, but cell-aggregates in BV smears from asymptomatic women were significantly smaller (median approximately 7,000 μm²) than those in no BV smears, p=0.0028.

We also compared the superficial cell index (SCI), a measure of cervicovaginal epithelial cell maturity, in no BV and BV smears from asymptomatic women with relatively low numbers of shed cells (≤50/100× field). The SCI of the no BV smears was significantly higher (median 0.86) than that of the BV smears from asymptomatic women (median 0.35), p=4.3e-98. This suggests a depletion of mature cells with exposure and shedding of underlying immature cells in BV with low number of shed cells, even in the absence of symptoms.

These results indicate that BV can contribute disproportionately to the increased susceptibility to reproductive tract infections associated with BV, without symptoms. These findings remained true when considering only those smears in which the microbiota comprised a diverse set of strict and facultative anaerobic bacteria (Community State Type IV (n=162), thus excluding those dominated by Lactobacillus spp. This is consistent with the hypothesis that high-shedding early stage BV in symptomatic women and low-shedding BV in asymptomatic women are not two separate forms of BV, but rather temporarily separated stages of the same condition. These findings can inform work on the clinical management of bacterial vaginosis, without having to rely on subjective reports of symptoms that do not provide a useful indication of the actual status of the condition or the susceptibility to STI or RTI and are not available in subjects that are not human

### 3. EMBODIMENTS OF THE INVENTION

A. Discussion

The equilibrium among the continuous processes of cell proliferation, maturation and shedding on the vaginal epithelium is probably essential to maintaining an effective barrier against pathogenic and non-pathogenic microbes alike. Here, we report an association of Stage I BV (roughly equivalent to Amsel-diagnosed “symptomatic” BV, which meets three of the four Amsel criteria and is accompanied by a subjective report of symptoms), with evidence of increased cell-shedding; and of Stage II BV (roughly equivalent to Amsel-diagnosed “asymptomatic” BV, which meets three of the four Amsel criteria and is not accompanied by a subjective report of symptoms) with both decreased cell-shedding and increased presence of immature epithelial cells. This information can be used to distinguish Stage I from Stage II BV so that diagnosed subjects can be aware of their status with respect to susceptibility to infection and can be afforded appropriate treatment for their condition, including treatment for previously untreated BV which is asymptomatic.

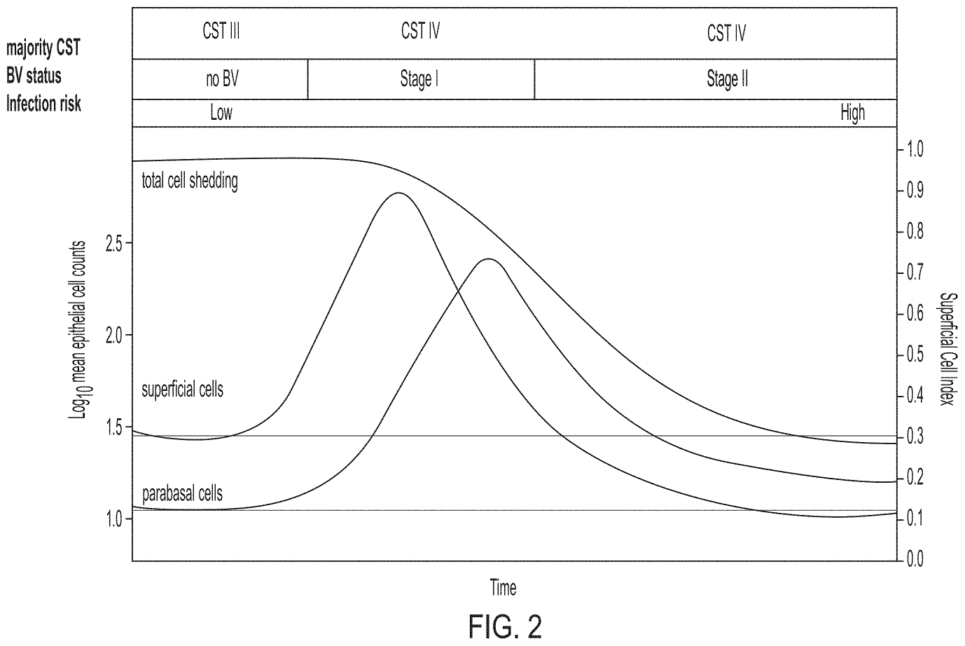

First, rather than sBV and aBV being two separate forms of BV, they sometimes can be two phases of a single condition. Under the model described here, where the old designations of “sBV” and “aBV,” Stage I BV may be rapidly followed by Stage II BV. In this case, the subject and/or the clinician may consider the subject cured and treatment stops, even though the subject still may be in increased danger of contracting further infections and should receive treatment or continue to receive treatment. See FIG. 2, which shows a time course of BV from lack of infection through Stage I to Stage II (approximate boundaries).

Under the disease course shown in FIG. 2, the increased cell-shedding events associated with Stage I exhaust the superficial and intermediate epithelial cell layers resulting in reduced cell-shedding, and exposure and loss of immature epithelial cells associated with Stage II. This hypothesis is supported by the finding that there are no significant differences in microbiota structure between Stage II and Stage I (high frequency of CST IV (93.94% and 76.47% respectively, p=0.205) and CST III (4.55% and 23.53% respectively, p=0.096), although the absolute abundances of bacteria have not been evaluated here and could be different between the stages.

Second and more importantly, the findings presented here suggest that Stage II BV is associated with more severe disruption of the vaginal epithelium than Stage I, and hence leads to a greater increase in susceptibility to infections. By analogy, the spermicide nonoxynol-9 (Hillier et al., 2005) is known to cause accelerated cell-shedding (Niruthisard et al., 1991), thinning (Vincent et al., 2011), disruption (Hoffman et al., 2004) and pro-inflammatory changes (Smith-McCune et al., 2015) on the vaginal epithelium, and was found to increase women's susceptibility to HIV (Wilkinson et al., 2002), as well as susceptibility to HSV-2 (Cone et al., 2006) and HPV (Roberts et al., 2007) in a mouse model. Stage II BV is associated with similar physiological effects on the epithelium.

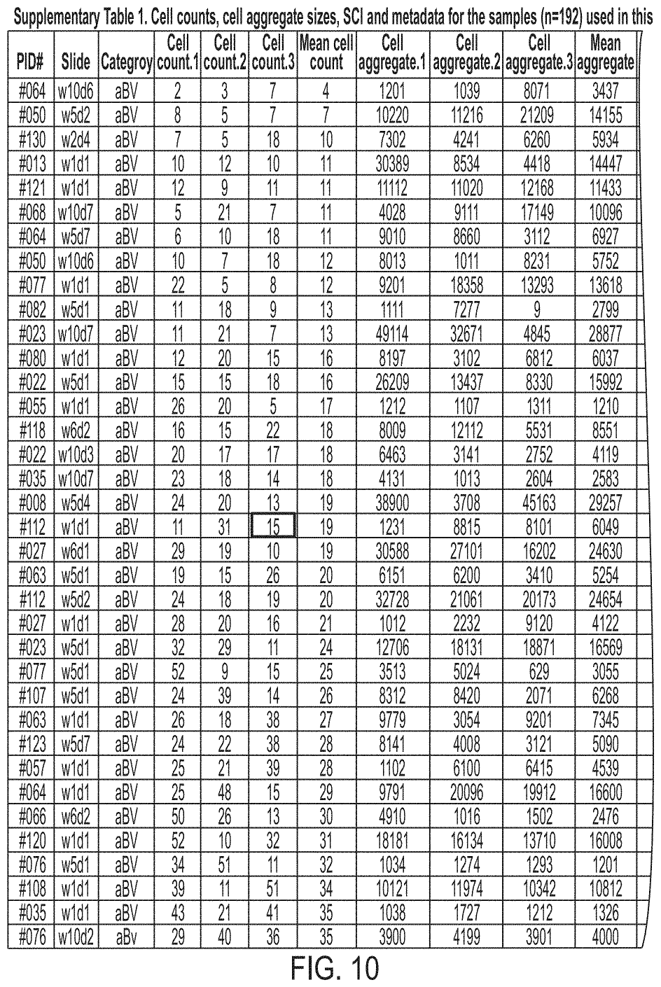

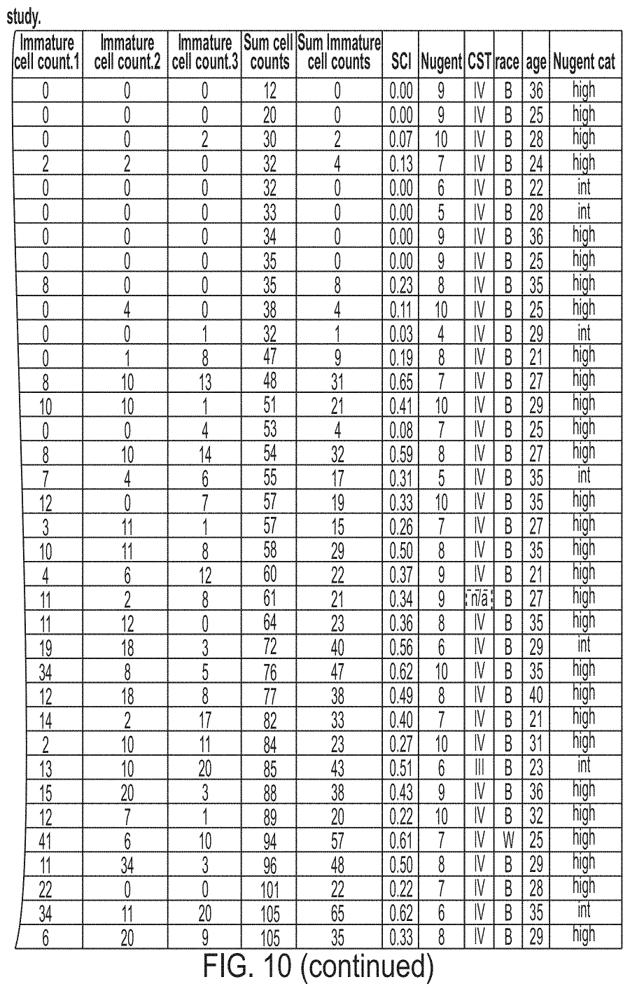

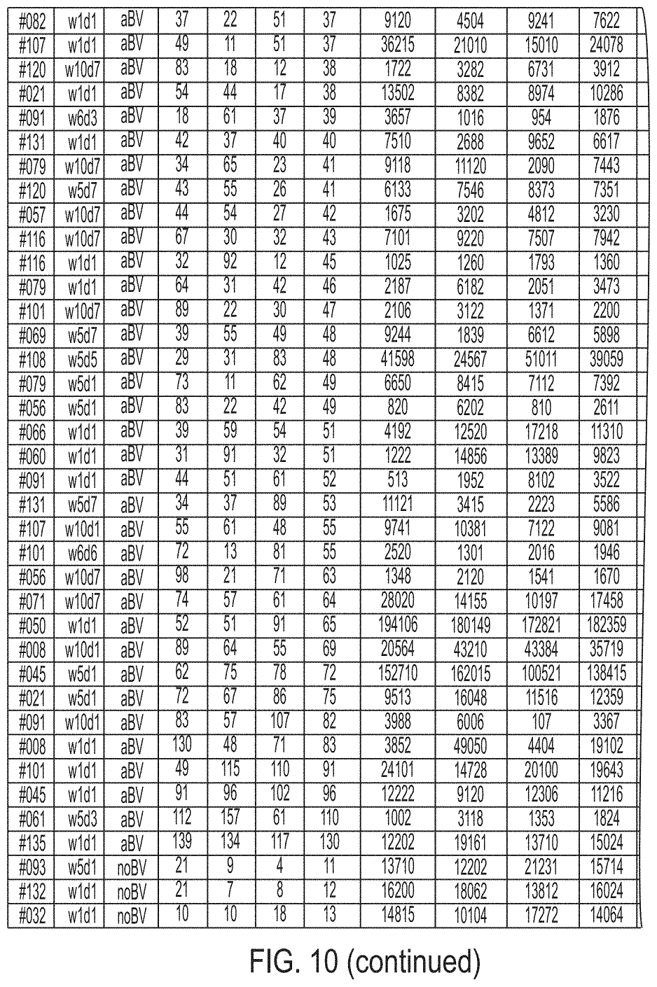

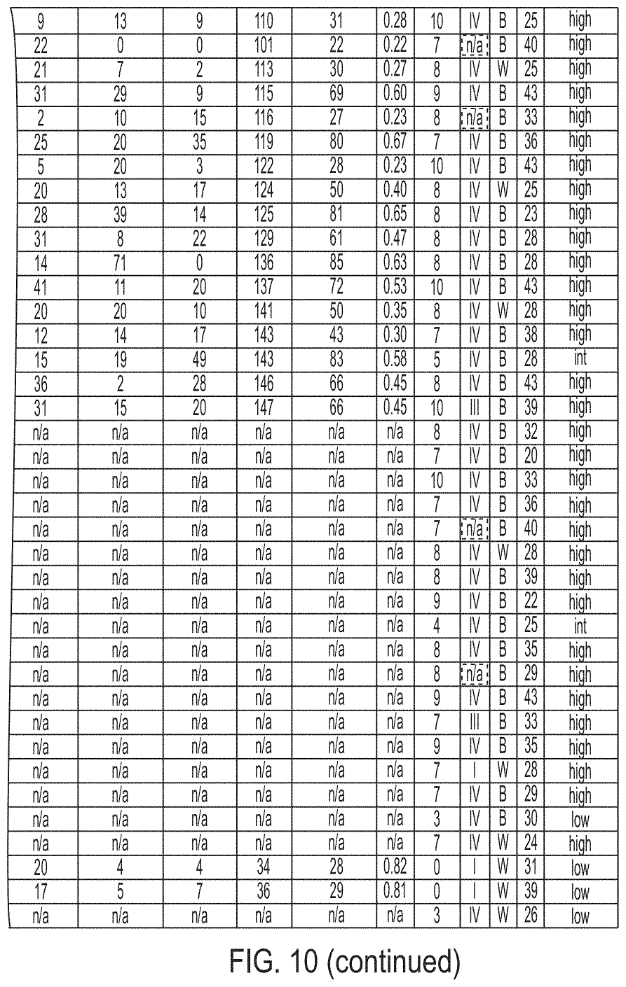

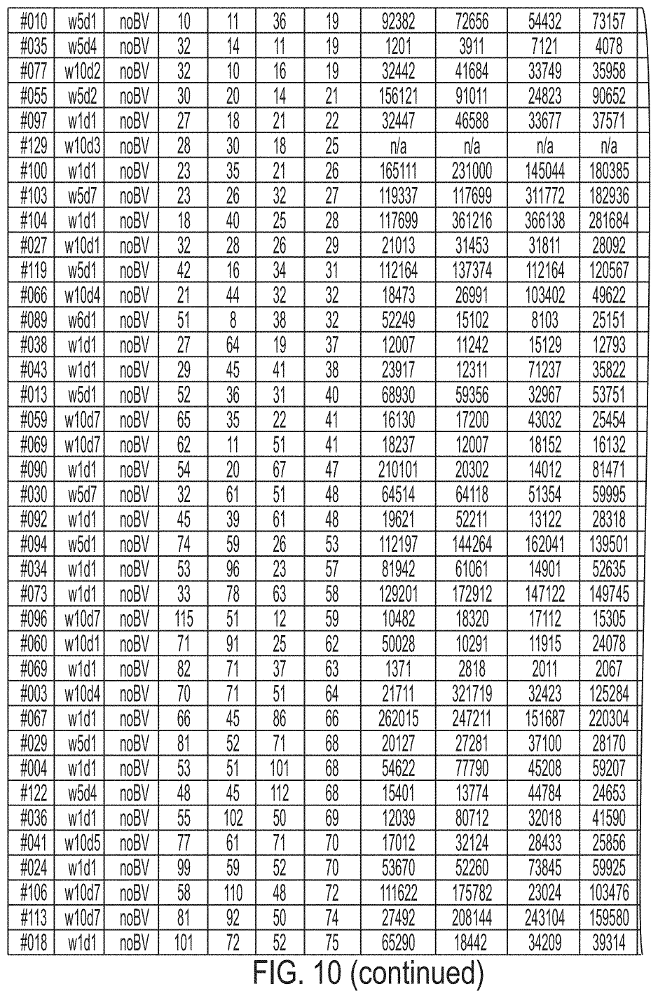

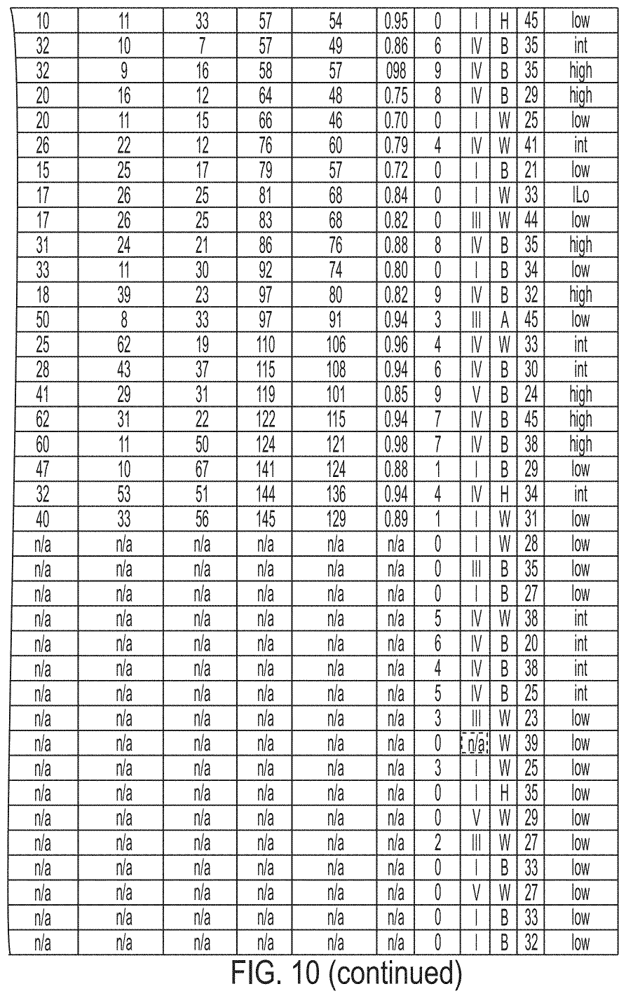

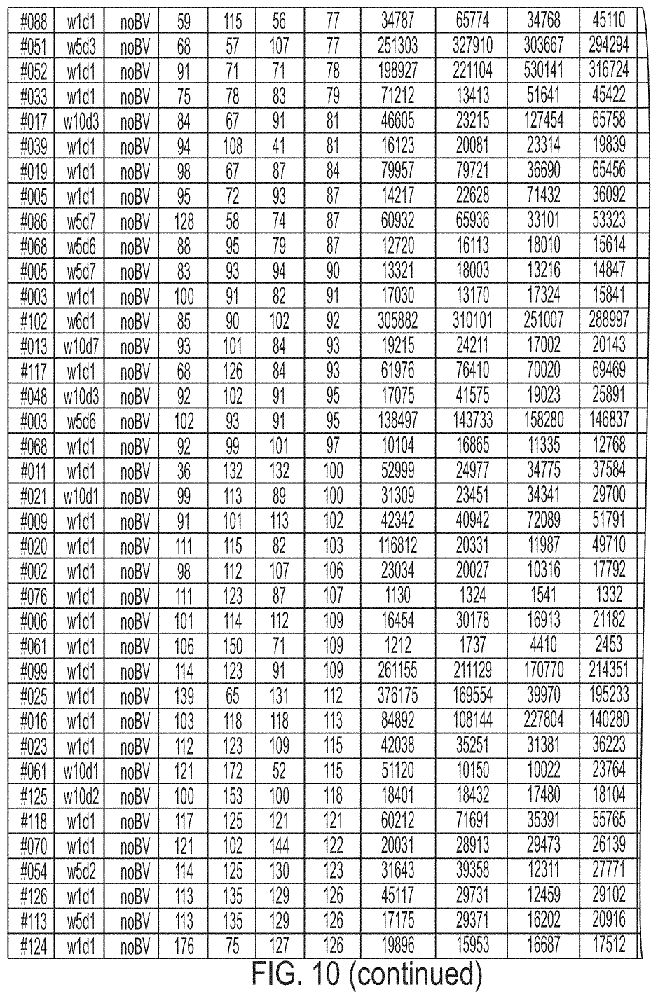

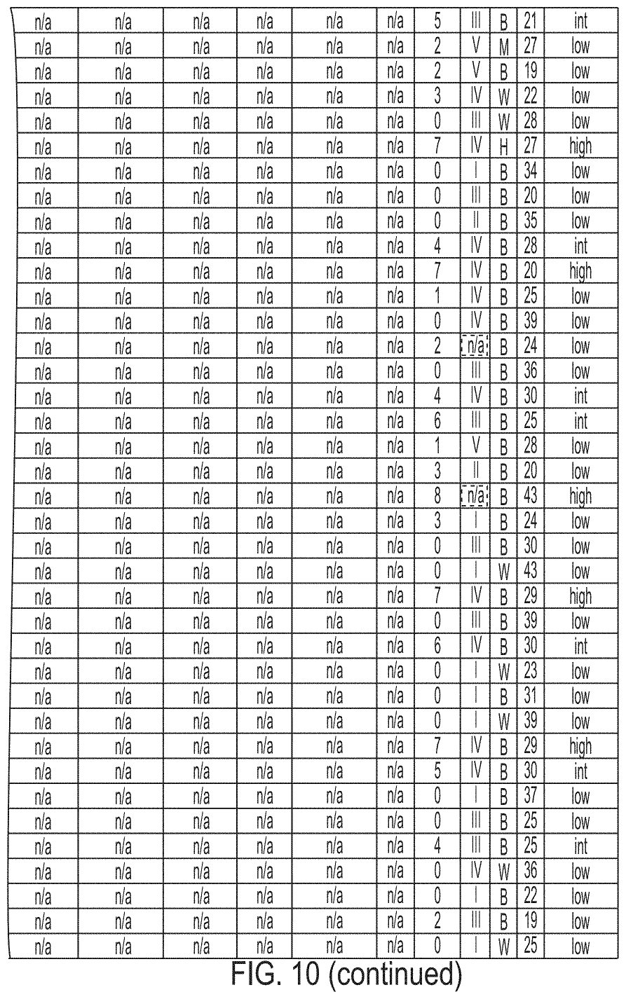

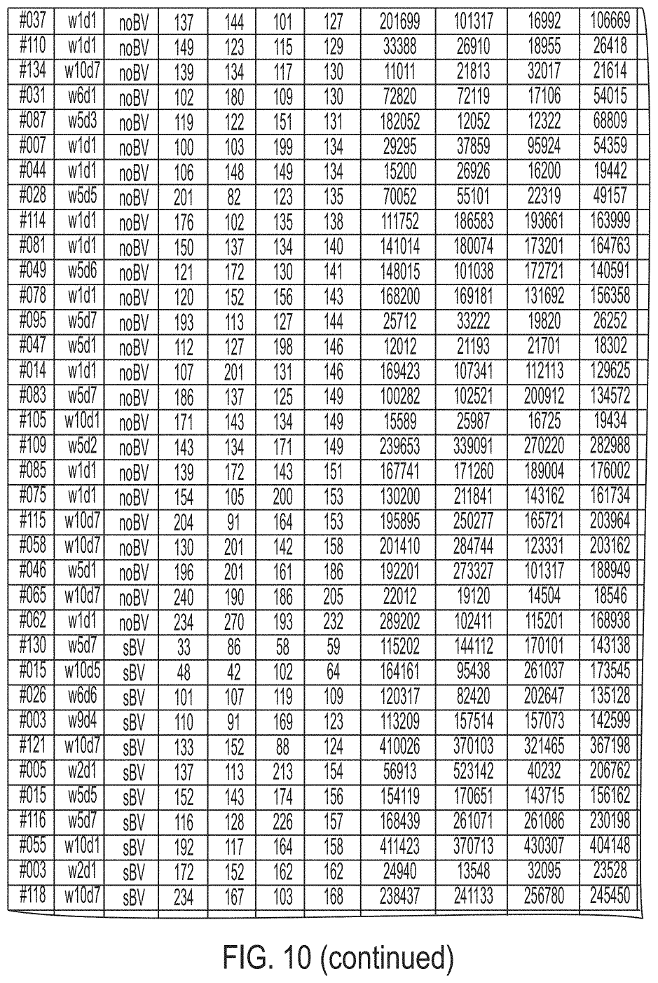

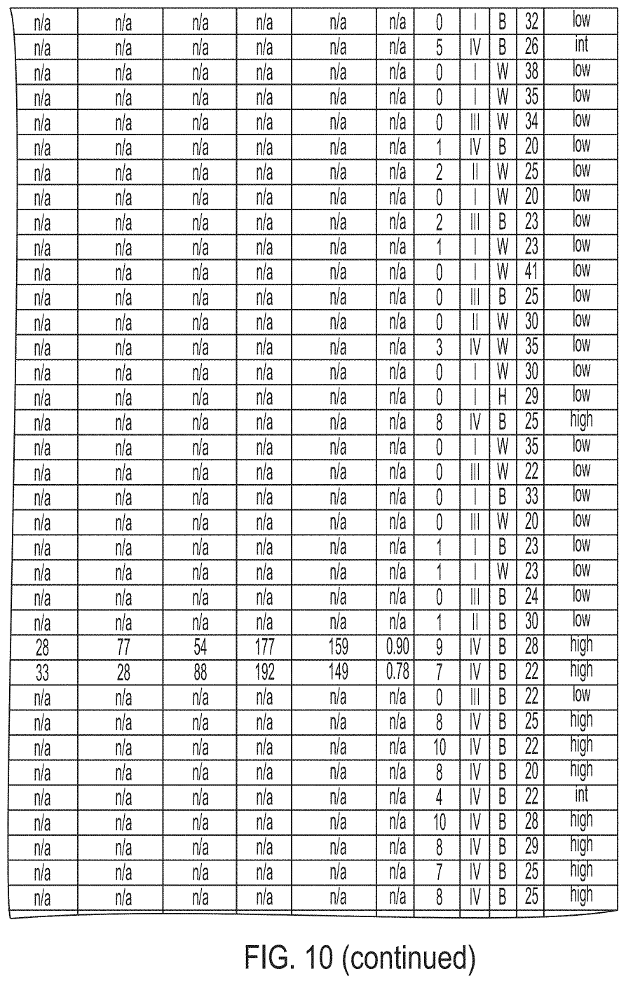

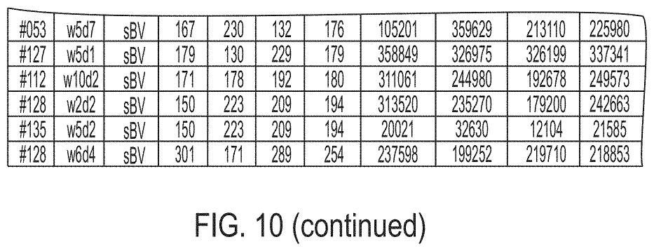

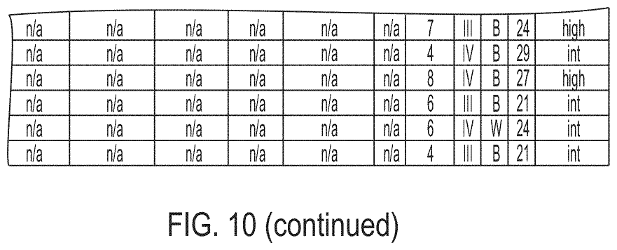

Stage II BV having a particularly disruptive effect on the cervicovaginal epithelium makes it necessary to reevaluate the relative importance of BV which is accompanied by symptoms and BV which is not, and the usefulness of the distinction between symptomatic and asymptomatic conditions. Stage I overlaps largely with the earlier, symptomatic phase of BV and phase II overlaps largely with the later, asymptomatic phase of BV, but these stages are largely reliant on subjective accounts by a patient, which can be unreliable. While vaginal symptoms have been linked in part to a patient's self-report (Klebanoff et al., 2004) thus symptoms perception, six women in this study presented with “sBV” and “aBV” at separate clinical visits over the course of the study (see FIG. 10).

Based on the findings here, there are relevant biological differences between Stage II and Stage I BV. Current CDC guidelines recommend antibiotic treatment only for BV with symptoms (Workowski and Bolan, 2015). The work presented here can lead clinicians to reconsider the wisdom of treating BV that is not symptomatic, and instead stage the BV and provide appropriate treatment for both Stage I and Stage II BV rather than merely counseling women with BV without symptoms to manage their increased susceptibility to infection, for example by avoiding unprotected sexual intercourse. However, in order to implement this, expanded screening based on high-resolution molecular assays of both the microbiota and the state of the epithelium would need to be implemented at routine gynecological visits, and more importantly, the development of specific therapies to address the epithelial disruption associated with Stage II BV would be needed.

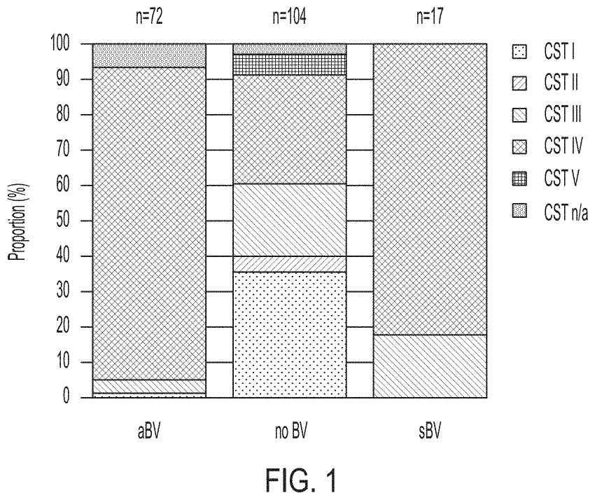

This application provides data showing that collected vaginal samples from symptomatic and asymptomatic women (earlier and later stage BV) differ significantly in the number and maturity of shed epithelial cells. Stage I BV samples from symptomatic Amsel-diagnosed women contain approximately one and a half times more epithelial cells than samples from women without BV; these Stage I samples contain superficial cells, intermediate cells, and parabasal cells. Stage II BV samples from Amsel-diagnosed women contain approximately one-third as many epithelial cells than samples from women without BV; these Stage II samples contain fewer superficial cells and more parabasal cells than samples from women without BV. See FIG. 1 and FIG. 2.

This discovery can be used to develop a laboratory-based or point-of-care BV diagnostic that is able to distinguish Stage I from Stage II BV. The platform would also be tremendously helpful in research, replacing time-consuming human-performed Nugent scoring. Combining the testing according to the invention with differential fluorescent staining of cell nuclei and cytoplasm can provide a single test that distinguishes Stage I and Stage II BV in a single test. Such a test would provide actionable information in the laboratory or at point-of-care for clinical uses and can open up enormous research possibilities by allowing Stage I and Stage II BV to be distinguished in previously collected vaginal samples.

Estimating the maturity of shed epithelial cells in a collected vaginal sample by microscopy is used to assess estrogenization of the cervicovaginal epithelium in menopausal women and in detection of estrus in breeding animals. This method depends upon the morphological changes in epithelial cells as they mature, beginning as spherical with a relatively large nucleus and narrow cytoplasmic space, and ending as squamous with a condensed nucleus and extensive cytoplasmic space. Normally, cells move from the basal layer through the intermediate layer to the superficial layer, from which they are shed.

B. Diagnostic Methods of the Invention

Existing methods for staging BV do not exist. Here, it has now been found that estimating the maturity of shed epithelial cells in a collected vaginal sample by microscopy can be used to determine the stage and severity of BV, whether the disease is overtly symptomatic or not. This method depends upon the morphological changes in epithelial cells as they mature, beginning as spherical with a relatively large nucleus and narrow cytoplasmic space, and ending as squamous with a condensed nucleus and extensive cytoplasmic space. Normally, cells move from the basal layer through the intermediate layer to the superficial layer, from which they are shed. Embodiments of the invention described herein take advantage of the newly discovered ability to distinguish the stages of BV using microscopy.

Therefore, certain embodiments of the invention relate to a method for diagnosing BV, and specifically distinguishing Stage I BV from Stage II BV to give helpful information to the clinician concerning whether treatment should be administered and what treatment(s) are appropriate for the stage of BV.

Testing as discussed above is performed as follows. The method involves obtaining a vaginal sample from a patient, preferably a mid-vaginal smear, and testing the sample for the number and maturity of shed epithelial cells. The diagnostic methods according to embodiments of the invention are useful to determine the presence of BV in the patient, and to determine the status and stage of the BV, Stage I or Stage II. Thus, performing a suitable battery of tests for appropriate bacteria, now will enable the clinician to discern the BV disease status of a patient with a clinical test alone, and will enable the clinician to administer an appropriate treatment more accurately and more quickly.

C. Tests/Systems/Kits

Certain embodiments of the invention, relate to diagnostic tests and testing kits. The present invention provides for kits for carrying out the above-described assays and methods. In certain embodiments, a kit according to the invention comprises components for collecting samples as are known in the art, appropriate containers, and equipment for performing the testing, such as slides, solutions and stains. Such kits would include, as nonlimiting examples, swabs for sample collection and containers for the swabs after a sample is collected, slides and appropriate solutions.

D. Treatments

Patients or subjects suitable for treatment or diagnosis according to embodiments of the invention can be any female mammal, including laboratory animals (e.g., mice, rats, rabbits, simians, and the like), zoo or endangered animals, companion animals (e.g., cats, dogs, and the like), farm animals (e.g., livestock such as horses, cows, sows, and the like), and the like, and including simians and apes (e.g., humans). The preferred subject is a human female.

Patients or subjects include any patient in need of diagnosis and/or treatment, and can easily be determined by the person of skill, such as a physician or other medical or research personnel in a clinic, hospital, research or clinical laboratory, and the like. Such patients include those with symptoms such as discharge, pain or itching, odor, or any suggestion of BV, past history of BV, or susceptibility to BV.

The samples to be collected from subjects that are useful for testing according to the invention are samples of vaginal fluids, vaginal smears or swabs, preferably a mid-vaginal smear. The ability to identify samples with low cell-count, low cell-maturity allows for a targeted treatment of the indicated condition. Treatments according to certain embodiments of the invention, for example, involve using a probiotic comprising Lactobacillus and Bifidobacterium strains chosen specifically to address epithelial disruption and depletion can be administered to the patient in need.

Some exemplary treatments include, but are not limited to:

1. Lactobacillus strain(s) with anti-inflammatory activity are contemplated for treatments to suppress expression of pro-inflammatory cytokines by stimulated epithelial cells. Epithelial disruption triggers an inflammatory response, which in turn causes further loss of structural integrity and barrier function.

2. Lactobacillus strain(s) with antioxidant activity are contemplated for treatments with antioxidants, through production of extracellular polysaccharides. Like inflammation, increased oxidative activity is both a consequence of epithelial disruption and a source of further damage.

3. Lactobacillus strain(s) that increase epithelial cell proliferation are contemplated for treatment of depletion of epithelial cells by producing cell proliferation. For example, a strain of Lactobacillus casei increases proliferation of intestinal epithelial cells and can do so also on the vaginal epithelium.

Alternatively or in addition, prebiotics are contemplated for use in treating BV. Any prebiotics that favor the growth of Lactobacillus species and/or restore the production of lactic acid (especially D-lactic acid), are contemplated to restore a healthy vaginal epithelium in subjects with BV.

Alternatively or in addition, symbiotics, comprising a mixture of probiotics and prebiotics can be used to treat BV. Any compound comprising nutrients or biochemical molecules that favor the growth of indigenous Lactobacillus, or those bacteria included in the probiotic formulation, are contemplated for use in treating BV.

Alternatively or in addition, antibiotics can be used to treat BV. Any antibiotics that would reduce or eliminate strict and facultative anaerobes, thus favoring the growth of Lactobacillus species are contemplated for use to restore a healthy vaginal epithelium in subjects with BV.

Alternatively or in addition, any molecules with antimicrobial properties (such as boric acid and the like, that are compatible with vaginal tissue) are contemplated to treat BV. These molecules These antibiotics can be used alone, in combination with, or followed by probiotic and/or prebiotic compositions to treat BV.

Preferred bacteria for treatment of BV include, but are not limited to Lactobacillus species such as L. casei, and various strains thereof. Probiotics which are contemplated for use with the invention include any probiotics known in the art. Prebiotics which are contemplated for use with the invention include any prebiotics known in the art.

Exemplary antibiotics that are contemplated for use in the invention include, but are not limited to any of the known antibiotics that can control the growth of undesirable or overgrown bacteria. Antimicrobial compositions that are contemplated for use in the invention include boric acid, and the like, or mixtures thereof.

Treatments according to the invention involving the administration of bacteria preferably are administered to the vaginal epithelium directly or indirectly. Any suitable method known in the art is appropriate and can be determined by the skilled physician or researcher. For example, a vaginal suppository or pessary can be used, or a liquid douche solution or emulsion, and the like, can be used. In addition, a tampon, cream, ointment, lotion, liposomes, or the like can be placed in the vaginal canal to administer bacteria.

Treatments according to the invention are contemplated to be administered in a dose of about 10⁵ to about 10¹¹ colony forming units (CFU), preferably about 10⁶ to about 10¹⁰ CFU, and most preferably about 10⁷ to about 10⁹ CFU for bacterial probiotics. Treatments according to the invention are contemplated to be administered in a dose of about 200 mg to about 1000 mg, preferably about 300 mg to about 900 mg, and most preferably about 400 mg to about 800 mg for prebiotics. Treatments according to the invention are contemplated to be administered in a dose of about 100 mg to about 500 mg, preferably about 150 mg to about 450 mg, and most preferably about 200 mg to about 400 mg, orally, every 6 hours for antibiotics such as clindamycin. Treatments according to the invention are contemplated to be administered in a dose of about 200 mg to about 1000 mg, preferably about 300 mg to about 900 mg, and most preferably about 400 mg to about 800 mg for antimicrobial compounds. However, these amounts can be adjusted by the skilled person depending on the patient's condition.

These treatments are contemplated for dosages that can include administration about 3-4 times daily, about twice daily, about daily, about every other day, about biweekly, about weekly, or about monthly and may continue for about 1 day, about 2 days, about 3 days, or more, including about 1 week, about 2 weeks, about 3 weeks, about 4 weeks, about 1 month, several months, or indeterminately.

### 4. EXAMPLES

This invention is not limited to the particular processes, compositions, or methodologies described, as these may vary. The terminology used in the description is for the purpose of describing the particular versions or embodiments only, and is not intended to limit the scope of the present invention which will be limited only by the appended claims. Although any methods and materials similar or equivalent to those described herein can be used in the practice or testing of embodiments of the present invention, the preferred methods, devices, and materials are now described. All publications mentioned herein, are incorporated by reference in their entirety; nothing herein is to be construed as an admission that the invention is not entitled to antedate such disclosure by virtue of prior invention.

### Example 1

### General Methods

### A. Clinical Samples

The current study used samples and data from the UMB-HMP study described in Ravel et al., “Daily temporal dynamics of vaginal microbiota before, during and after episodes of bacterial vaginosis.” Microbiome 1, 29. 2013, the contents of which are incorporated by reference in their entirety. A total of 135 non-pregnant, reproductive-aged women took part in a 10-week observational longitudinal study during which participants self-collected daily vaginal samples under a protocol approved by the Institutional Review Boards of the University of Alabama at Birmingham and of the University of Maryland School of Medicine. Written informed consent was obtained from all participants.

The study included examination by a clinician at enrollment, week 5 and week 10, or at interim times if vaginal symptoms were reported. None of the samples used in this study were positive for STI or yeast infection. The clinician's record for each examination included a diagnosis of BV performed according to the Amsel criteria (Amsel et al., 1983). For these samples, a diagnosis of “symptomatic BV” was established when the participant reported symptoms on direct questioning and fulfilled at least three of the four Amsel criteria; a diagnosis of “asymptomatic BV” was established when the participant did not report symptoms but fulfilled at least three of the four Amsel criteria.

Each day, participants self-collected a vaginal swab, smeared it on a microscope slide and placed the slide in a protective sleeve. Participants dropped off the slides to the clinic every week. The slides were Gram-stained and scored for BV using the methods of Nugent (see Nugent et al., “Reliability of diagnosing bacterial vaginosis is improved by a standardized method of gram stain interpretation.” J Clin Microbiol 29, 297-301, 1991, the contents of which are hereby incorporated by reference in their entirety) by three independent readers (Ravel et al.,2013). Another daily swab was stored in Amies transport medium and immediately frozen at −20° C. This swab was used for extraction and purification of genomic DNA using a QIAsymphony™ robotic platform and QIAGEN CellFree 500 kits (QIAGEN, Valencia Calif.); metataxonomic analysis (Marchesi and Ravel, 2015). The V3-V4 hypervariable regions of bacterial 16S rRNA genes were amplified and sequenced on an Illumina MiSeg™ instrument to obtain the bacterial composition and abundance of each sample according to known methods (Fadrosh et al., 2014). Sequencing was performed at the Institute for Genome Sciences' Genomic Resource Center (GRC) at the University of Maryland School of Medicine (marylandgenomics.com). CST (Gajer et al, 2012) were assigned using VALENCIA, a novel nearest centroid classification algorithm based on the classification of an over 13,000 vaginal microbiota dataset. This approach to CST assignment is more robust than standard within-study hierarchical clustering and allows for between-studies comparisons (Ravel, 2018).

### B. Microscopy

Vaginal smears collected on the day of clinical examination (study entry, week 5 and week 10 for each participants) were evaluated as described below and the findings evaluated with respect to associated clinical and microbiota data.

A total of 192 smears from 126 women met the criteria above and were available to be examined. Following the method previously described to evaluate cervicovaginal epithelial cell-shedding (Amegashie et al., 2017), slides were visualized using a Zeiss™ Plan-ACHROMAT 10× objective on a Zeiss™ Primo Star microscope (Carl Zeiss™ Microscopy LLC, Thornwood N.Y.) for a total magnification of 100×, and images of three representative fields were captured from each slide using a Zeiss™ Axiocam ICc3 camera and software.

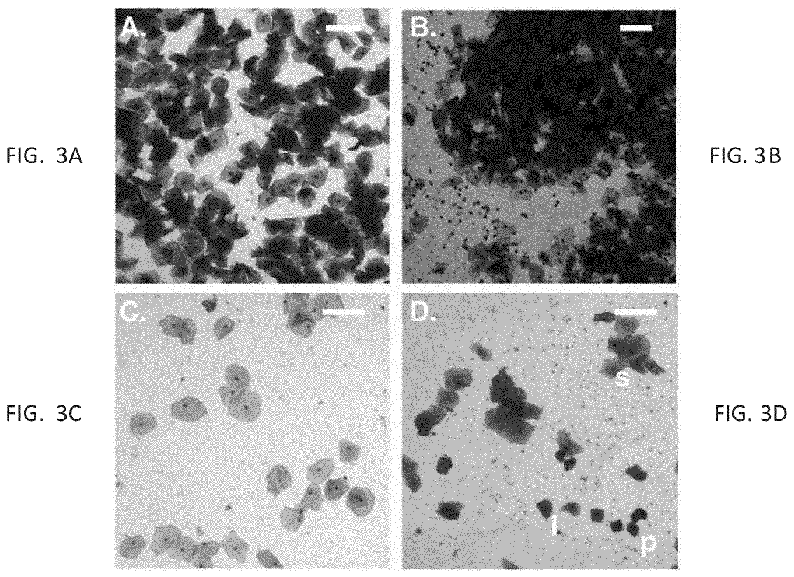

Representative fields were located within the main body of the smear, not near the start, end, or margins; representative fields were chosen to exclude aggregations of epithelial cells too dense to distinguish cells. All complete epithelial cells in the three images were counted (i.e. cells falling partly outside the image were not counted) and the mean count calculated. Images of three representative cell-aggregations were captured, also from locations within the main body of the smear, away from the start, end, or margins. Cell-aggregation size was measured using the ‘measure>outline’ tool of the imaging software (unit μm²) (AxioVision™ v 4.8.2.0); the mean cell-aggregation size was calculated. When the mean epithelial cell count was ≤50, the three captured images were further analyzed, counting the number of superficial cells (cells with a condensed nucleus, large cytoplasmic space, and polygonal shape with thin, angular margins). From this, the superficial cell index (SCI=number of superficial cells/number of all epithelial cells) was calculated (range 0.0-1.0) according to the methods of Stupnicki, “A dynamic approach to the evaluation of oestrogenic effects on the vaginal epithelium in women.” J Reprod Fertil 22, 563-567R, 1970. A SCI of 1.0 corresponds to high numbers of mature cells and low or no immature cells, while a low SCI corresponds to low numbers of mature epithelial cells and high numbers of immature epithelial cells. An example of different types of smears is shown in FIG. 3. All measurements are listed in FIG. 10. The dataset presented here in FIG. 10 is effectively cross-sectional and contains only 7 women who provided both early “sBV” samples and “aBV” samples, collected several weeks apart.

### C. Statistical Analysis

Cell counts, cell-aggregate sizes, and SCI values were reported as median and interquartile range. Comparisons of cell counts were performed by fitting a Bayesian Laplace subject-wise random effects model to the log10 transformed data. Comparisons of cell-aggregate sizes were made in the same way. SCI values were computed within a Bayesian binomial mixed effects model with subject-wise random intercept. Adjustment for multiple testing was performed using false discovery rate (FDR). For all comparisons, exact p values are reported. Within woman pairwise comparison of log10 cell counts and aggregation sizes was made using a Bayesian Poisson within subject two group comparison model. All scripts used in this study are available on GitHub (see github.com/ravel-lab/BV_CELL_SHEDDING).

### Example 2

### Representative Bright-Field Microscopic Images

Photomicrographs were prepared of representative fields from vaginal smears. FIG. 3 is a set of bright-field micrographs (100× total magnification) showing representative samples. FIG. 3A shows a no BV (by Amsel analysis) sample with a cell count >100/100× field, showing predominantly superficial cells shed as singletons. FIG. 3B shows a BV sample from a symptomatic subject (by Amsel analysis) with a cell count >100/100× field, showing predominantly superficial cells shed in large aggregates. FIG. 3C shows a no BV (by Amsel analysis) sample with a cell count <50/100× field, showing predominantly superficial cells. FIG. 3D shows a BV sample from an asymptomatic subject (by Amsel analysis) with a cell count <50/100×, showing superficial cells (example marked s) shed in combination with intermediate (example marked i) and parabasal (example marked p) cells.

### Example 3

### Comparison of Total Epithelial Cell Counts

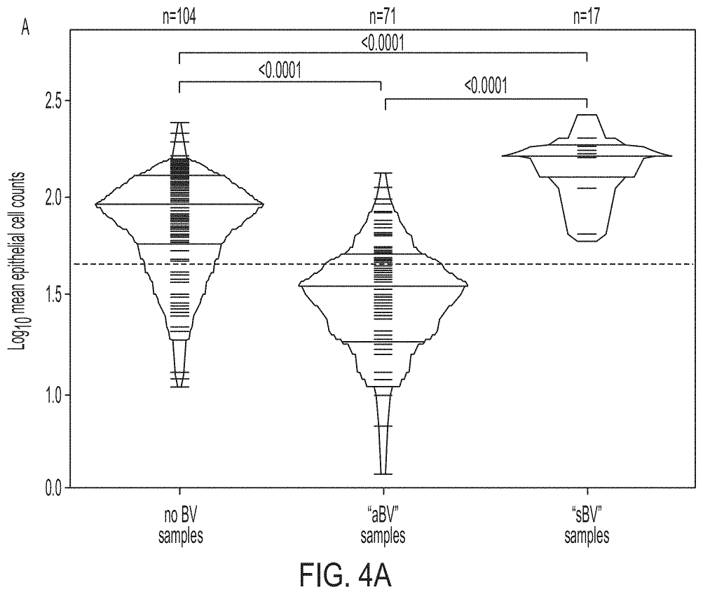

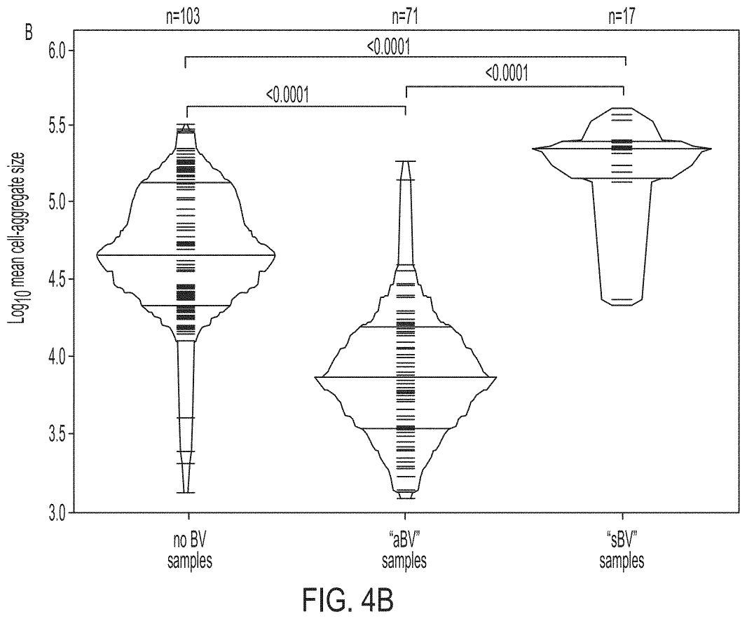

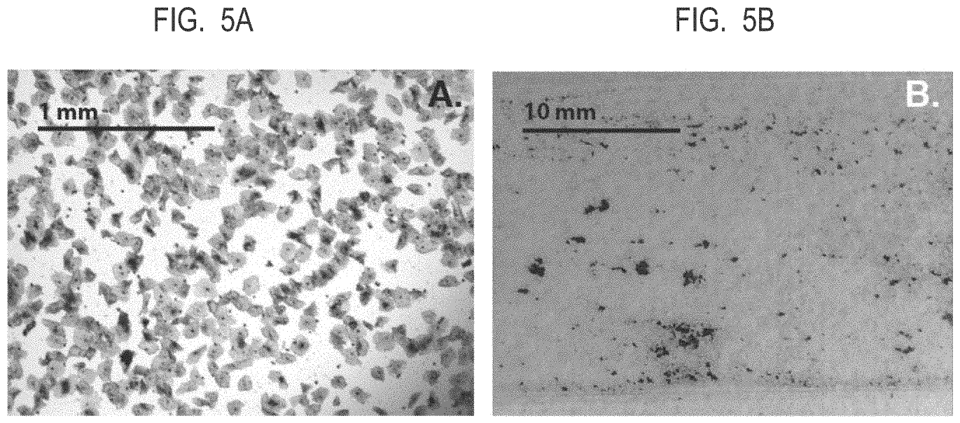

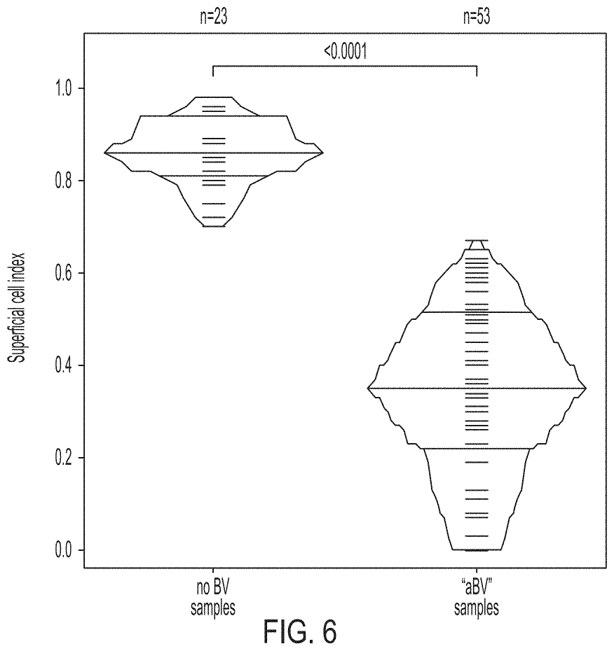

Cell counts were first considered based on the prior diagnostic group (noBV, “sBV,” or “aBV” based on Amsel criteria) only, without reference to microbiota CST information, and are referred to here using this nomenclature, here and in the examples below. The median cell count of samples with no diagnosis of BV (noBV, n=104) was 91/100× field (interquartile range 58-126). This was significantly lower than the median cell count for samples with a diagnosis of “sBV” (“sBV,” n=17}, which had median 158/100× field (IQR 124-179), p=7.2e-09. Additionally, the median cell count of noBV samples was significantly higher than that of samples with a diagnosis of “aBV” (“aBV,” n=71), which had a median 35/100× field (IQR 19-50), p=2.4e-16. See FIG. 4A. In addition, median cell counts of the “sBV” and “aBV” samples were significantly different, p<10e-12.

### Example 4

### Comparison of Cell-Aggregates Size

Variability of cell-aggregates on the slides was apparent. Some slides had evenly dispersed cells with aggregates of only a few cells each (see FIG. 5A), while other slides had multiple aggregations so extensive and dense that they could be distinguished without magnification (see FIG. 5B). Cell-aggregate size measurements indicated that in noBV samples, the median cell-aggregate size (50,000 μm², IQR 20,000-140,000 μm²) was significantly smaller than that for “sBV” samples, which had a median cell-aggregate size of 220,000 μm² (IQR approximately 140,000-240,000 μm²), p=1.5e-14. Additionally, the median cell-aggregate size of noBV samples was significantly larger than that of a BV samples, which had a median cell-aggregate size of approximately 7,000 μm² (IQR 3,000-14,000 μm²), p<2.22e-16 (see FIG. 4B). Lastly, the median cell-aggregate sizes of “sBV” and “aBV” samples were significantly different, p=2.2e-55.

### Example 5

### Comparison of the Superficial Cell Index

The superficial cell index (SCI) was calculated for samples with a mean cell count of 50/100× field, corresponding to a total of 78 samples from 54 women. Mean cell count of 50/100× field was chosen since it included almost all “aBV” samples and was well-represented among noBV samples. The median SCI of noBV samples (n=23) was 0.86 (IQR 0.81-0.94), significantly higher than that of “aBV” samples (n =53), which had a median SCI of 0.35 (IQR 0.22-0.51), p=4.3e-98 (see FIG. 6). No “sBV” samples had mean cell count of 50/100× field.

### Example 6

### Comparison of Total Epithelial Cell Counts, Cell-Aggregate Sizes and SCI Stratified by Amsel Diagnostic Groups and CSTs

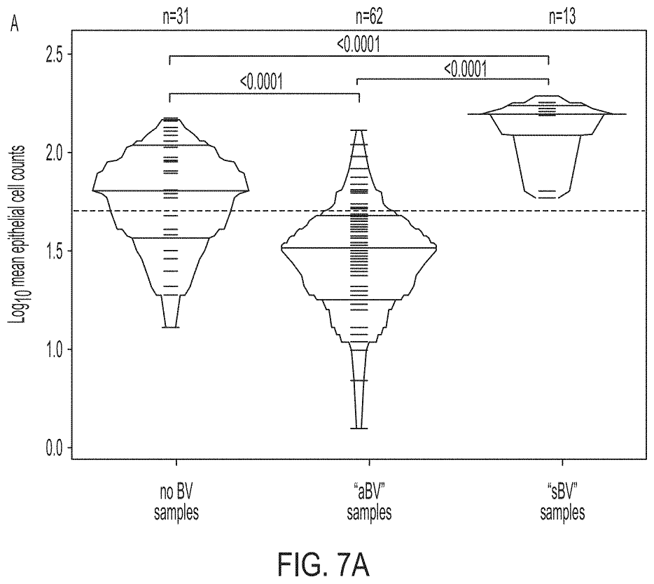

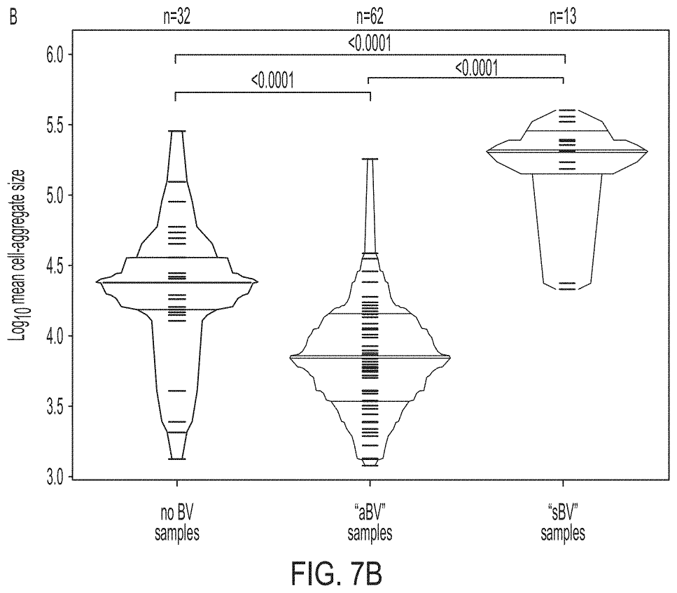

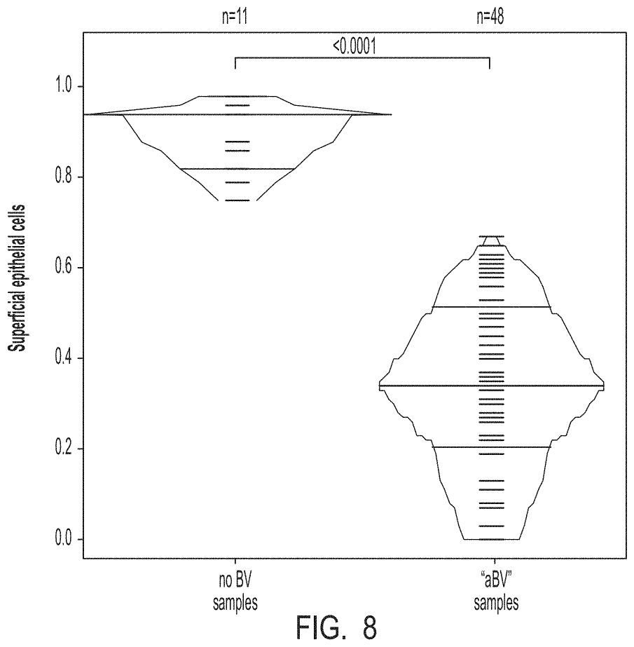

Interestingly, differences between noBV, “sBV,” and “aBV” remained when samples were considered based on Amsel diagnostic groups and CSTs. Among samples assigned to CST IV (microbiota comprising a wide range of facultative and strict anaerobic bacteria and a lack of Lactobacillus spp.), the median cell count of (1) noBV samples, (64/100× field, IQR 38-112), n=31, (Amsel-BV negative, but molecular-BV positive sample) was significantly lower than that of “sBV” samples (157/100× field, IQR 157-171, n=13), p=9.5e-7, and significantly higher than “aBV” samples (33/100× field, IQR 18-48), n=62), p=0.00011 (see FIG. 7A). In addition, median cell counts of “sBV” and “aBV” samples assigned to CST IV were significantly different, p=1.3e-24. Similarly, the median cell-aggregate size of CST IV noBV samples (20,000 μm², IQR 15,000-25,000 μm²) was significantly smaller than that of CST IV sBV samples (25,000 μm², IQR 15,000-36,000 μm², p=5.8e-16,and significantly larger than that of aBV samples (7,000 μm2, IQR 3,600-13,000 μm2, p=1.0Ge-9 (see FIG. 7B). Further, the median cell-aggregate sizes of “sBV” and “aBV” samples assigned to CST IV were significantly different, p=3.15e-45. Lastly, among the 6lsamples with a mean cell count of ≤50/100× field that were classified as CST IV, the median SCI of noBV samples (0.94 IQR 0.84-0.95), n=11) was significantly higher than that of “aBV” samples (0.34, IQR 0.22-0.51 n=48), p=5.3e-73 (see FIG. 8). There were too few samples with a microbiota dominated by Lactobacillus spp. that were categorized as “sBV” (n=4, all CST Ill) or “aBV” (n=4, one CST I and three CST Ill) to permit any analysis for CSTs other than CST IV.

### Example 7

### Median Cell Count of noBV Samples Assigned to CST IV

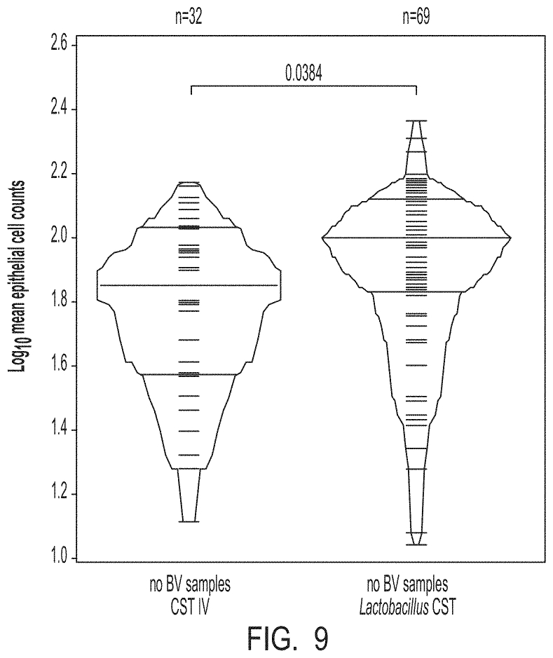

The median cell count of noBV samples assigned to CST IV (70/100× field, n=32) was significantly lower than that of other noBV samples (93/100× field, n=69), p=0.0384. See FIG. 9. This is likely because molecular-BV broadly defines BV and is not limited to Amsel-diagnosed “sBV” and Amsel-diagnoses “aBV” (as evidenced by the data set, in which 31/104 noBV samples were CST IV). Interestingly, the BV-associated increased risk of HIV acquisition is higher in studies using molecular-diagnosed BV (defined as all forms of CST IV) as the exposure variable, compared to studies using Amsel-diagnosed BV or Nugent-diagnosed BV (McKinnon et al., 2019).

A recent meta-analysis showed that Amsel-diagnosed BV was associated with a 2-fold increase in risk for HIV acquisition (OR: 1.93, 95% CI: 1.45-2.57) (Atashili et al., 2008). A subsequent study suggested that molecular-diagnosed BV was associated with a 4-fold hazard ratios compared to L. crispatus-dominated samples (HR:4.41, 95% CI: 1.17-16.61, p=0.028) (Gosmann et al., 2017). Molecular-diagnosed BV is associated with proinflammatory cytokines (Gosmann et al., 2017) and activated HIV-target cells (Anahtar et al., 2015) in the cervicovaginal epithelium and thus with increased risk of HIV acquisition.

These findings support a higher risk associated with molecular-diagnosed BV in asymptomatic women, and suggest mechanisms involving impaired physiological state of the epithelium and increased pro-inflammatory markers. Epidemiologic studies of BV and risk for HIV suggest that molecular-diagnosed BV might trend toward higher point estimates for HIV risk than Amsel-diagnosed BV because molecular diagnosis includes more women with at-risk Stage II BV states. To our knowledge, no studies have directly assessed the risk of HIV or other STI acquisition in women with molecular-diagnosed BV versus Amsel-diagnosed “sBV.”

### Example 8

### Raw Counts and Metadata

The raw counts and metadata used in this study are available in FIG. 10.

### Example 9

### Conceptual Model

FIG. 2 shows a graph illustrating the time-course of BV stages. Under these conditions, the increased cell-shedding events associated with Stage I BV exhaust the superficial and intermediate epithelial cell layers, eventually resulting in reduced cell-shedding, and exposure and loss of immature epithelial cells (low SCI) associated with Stage II BV. This progression is likely to be associated with increased risk of infections, including sexually transmitted infections.

### Example 10

### Diagnosis

A suitable vaginal smear is taken and subjected to cell counting and/or obtaining a superficial cell index. Diagnostic criteria for no BV, Stage I BV, and Stage II BV are contained in Table 2 and based on FIG. 2.

TABLE 2
Diagnostic criteria.

| | |
|---|---|
| No BV: | total shedding cell count above about 2.8 log10 |
| | Superficial cell count below about 1.8 log10 |
| | Parabasal cell below about 1.1 log10 |
| Stage 1: | total shedding cell count from about 2.4 log10 |
| | to about 3.0 log10 |
| | Superficial cell count from about 1.5 log10 |
| | to about 2.7 log10 |
| | Parabasal cell count from about 1.1 to about |
| | 2.5 log10 |
| Stage II: | total shedding cell count from about 1.5 log10 |
| | to about 2.5 log10 |
| | Superficial cell count from about 1.0 log10 |
| | to about 1.7 log10 |
| | Parabasal cell count from about 1.2 log10 |
| | to about 2.3 log10 |

1. A method of diagnosing the stage of bacterial vaginosis in a subject, comprising:
(a) obtaining a sample of vaginal secretion from the subject;
(b) determining the total number of shed epithelial cells in the sample; and
(c) determining the maturity of shed epithelial cells in the sample to obtain epithelial cell counts for superficial cells and for parabasal cells in the sample to diagnose Stage I or Stage II of bacterial vaginosis,
wherein Stage I bacterial vaginosis is diagnosed when total cell shedding log10 cell count is from about 2.4 to about 3.0, superficial cells log10 cell count is from about 1.5 to about 2.7 and parabasal cells log10 cell count is from about 1.1 to about 2.5; and
wherein Stage II bacterial vaginosis is diagnosed when total cell shedding log10 cell count is from about 1.5 to about 2.5, superficial cells log10 cell count is about 1.0 to about 1.7 and parabasal cells log10 cell count is about 1.2 to about 2.3.

2. A diagnostic kit for diagnosing the stage of bacterial vaginosis comprising means for performing the method of claim 1.

3. A method of treating bacterial vaginosis in a subject in need, comprising:
(a) performing the method of claim 1; and
(b) treating the subject for bacterial vaginosis if the subject is diagnosed with Stage I BV and treating the subject for bacterial vaginosis if the subjects is diagnosed with Stage II BV.

4. The method of claim 3, wherein the subject is asymptomatic.

5. The method of claim 3, wherein the subject is symptomatic.
